(19) United States
(12) Patent Application Publication
(10) Pub. No.: US 2005/0234381 A1
(43) Pub. Date: Oct. 20, 2005

(54) Method for allowing operator entries at a medical instrument

(76) Inventors: Guenter Niemetz, Melsungen (DE); Rolf Hollstein, Alheim (DE); Dirk Moeller, Altmorschen (DE); Ulrich Wenzel, Melsungen (DE); Gerhard Bock, Friedewald (DE); Rolf Heitmeier, Baunatal (DE)

Correspondence Address: RATNERPRESTIA, P O BOX 980, VALLEY FORGE, PA, 19482-0980, US

(21) Appl. No.: 11/080,251

(22) Filed: Mar. 15, 2005

(30) Foreign Application Priority Data
Mar. 30, 2004 (DE) 10 2004 013 814.1

(51) Int. Cl. A61M 37/00

(52) U.S. Cl. 604/4.01

(57) ABSTRACT

A medical instrument, in particular a machine for extracorporeal blood treatment, allows for execution of different programs with selectable program parameters. The medical instrument (11) comprises a read unit (14) which is capable of communicating with a transponder (15) carried along by the personnel. Said transponder (15) contains an identification (ID). In dependence on the identification, different access authorizations with regard to selecting programs and/or modifying program parameters are granted. In this manner, it is determined what settings and/or modifications may be carried out by the nursing personnel and what settings and/or modifications may be carried out by the technical service personnel.

### BACKGROUND OF THE INVENTION

1. Field of the invention

The invention relates to a method for allowing operator entries at a medical instrument which can execute programs with selectable program parameters, and a medical instrument with the aid of which such a method can be carried out.

1. Description of related art

Medical instruments, to which the method according to the invention is applicable, are in particular blood treatment instruments for extracorporeal blood treatment and special dialysis instruments, such as described in EP 0 623 357 A1 or DE 197 42 633 C2. Such instruments comprise a computer with a screen and an input device with keys, switches, buttons or a membrane keyboard. Further, the input device can be integrated into the screen if the latter is configured as a touch screen. The input device allows user entries to be made for the purpose of selecting modes of treatment, setting operating parameters or acknowledging an alarm.

US 2003/0212314 Al describes a medical instrument which determines the presence of a person in front of the medical instrument with the aid of a detector. The parameters of the medical instrument can be adjusted only when a person is present in front of the instrument. In this manner, unintended adjustments caused by ambient influences can be prevented to a large extent.

In the field of instrument-aided medical treatment, the operating personnel carry out a treatment tailored to the patient by selecting various treatment programs, treatment parameters or alarm-acknowledging functions. Conventional operating units, such as touch screens, have the drawback that unintended touching can easily result in a change of programs or parameters. For example, an undesired contact cannot be reliably precluded when the input device is cleaned with a wipe or a cloth. In particular, faulty operations caused by unintended contact are difficult to perceive since the operating and display fields of touch screens are small, which may have serious consequences for the patient when important parameters are involved.

Conventional access control mechanisms, such as inquiry of a password (DE 2 146 074), identification cards with magnetic strips (WO 87/02160) or bar-code scanning systems (U.S. Pat. No. 5,384,756) have serious drawbacks for the user or with regard to the treatment. The drawbacks encountered in inquiry of the password are the long duration of the manual entry of the password, the limitation to numerical codes and the possibility of faulty entry of the password in stress situations. Magnetic identification cards suffer from mechanical wear of the magnetic strip during the reading process, and mechanical stress when the card is carelessly handled. Optical scanning systems for bar-code read devices have similar drawbacks as magnetic identification cards. In addition, they required direct visual contact between transmitter and receiver.

WO 87/02160 describes a computer containing medical files of patients. Here, an access control system offering limited access to various groups of personnel is provided. Said limited-access system merely requires fetch of data from the electronic file.

### SUMMARY OF THE INVENTION

It is an object of the present invention to provide a method for allowing operator entries at a medical instrument, which is suitable for carrying out a medical treatment of the body of a patient according to predetermined programs using selectable program parameters, wherein an operator identification is entered into a computer controlling said medical instrument, and wherein, in dependence on the operator identification, different access authorizations for selecting programs and/or modifying program parameters and/or entering alarm acknowledgements are granted, and which allows persons with different qualifications to carry out respective operator tasks.

According to the invention, it is provided that an operator identification is entered into a computer controlling the medical instrument, and, in dependence on the operator identification, different access authorizations are granted for selecting programs and/or modifying program parameters and/or entering alarm acknowledgements.

The invention offers the advantage that an access control can be effected with the unique operator identification in a multistage hierarchy system. An authorization for entering treatment parameters is granted in dependence on the operator identification of the respective operator. Classification of the operator identification may be effected such that the nursing staff of a dialysis ward are granted access to therapy-relevant parameters, such as starting or ending of therapy and alarm acknowledgement. Once the computer has determined the identification of a person belonging to the service personnel (service technician), said person, in addition to the access authorization for the nursing personnel, is granted authorization to set configuration and service parameters. Thus, the invention allows for granting of different authorizations of access to different programs and/or functions as well as different authorizations for activating or modifying said programs and/or functions.

In different treatments, such as dialysis, therapy records are manually prepared for each treatment. The invention allows such a record to be automatically prepared. The medical instrument generates a data structure in its data memory, which data structure corresponds to the manually kept record. The instrument stores all patient data, such as name, identification number, weight and set therapy values, such as treatment time, quantity of collected ultrafiltrate, conductivity of the haemodialysis solution. In addition to the stored data, the current operator identification is stored which is transmitted from a read unit at the time of data entry. In this manner, the record includes information on the identity of the operator who has carried out the setting and monitored the treatment.

Suitable data carriers for operator identification are conventional data carriers, such as magnetic cards, bar-code data carriers or chip cards. Further, it is possible to provide an identification in the form of a password or an ID number which is manually entered by the operator into the input device. Preferably, the operator identification is contained in a transponder carried along by the operator, which transponder contactlessly communicates with a read unit of the medical instrument. Such a solution is advantageous in that a transponder is easy to operate and the identification takes little time. A transponder is an instrument which contactlessly responds to an inquiry by the read unit connected with the computer, and supplies a digital signal in response to such an inquiry, said signal identifying the person involved. Normally, transponders do not require their own energy supply unit. An example of a transponder which ensures compatibility of the components of a medical instrument is described in DE 20113789 of the same applicant.

When transponders are used which comprise corresponding memories, it is also possible that the read unit, which in this case is configured as a write/read unit, reads the respective record into the transponder. Thus, at the end of the treatment, the record can be stored in the transponder of the operator with a view to later entering it into a different computer system which also comprises a read unit for the transponder. Here, another data processing is carried out for documentation and quality assurance purposes. The same aim can be achieved when the medical instrument transmits, continuously during the treatment or at the end of the treatment, the record together with the operator identification by means of data communication to a different computer system. The record can also be directly printed out at the medical instrument.

Further, it is possible to assign a patient identification to each patient, and equip each patient with a transponder in which this identification is stored. The patient identification includes information on the fact that the person concerned is a patient (and not an operator). If the transponder comprises an adequately large memory, it is possible to read the treatment record, including the operator identification, into a patient's transponder. In this manner, the patient carries along in the transponder the records of previous treatments. Such a system offers the possibility of storing in the transponder the treatment parameters for subsequent therapies. Directly before the next treatment, these treatment data are read by the read unit of the medical instrument and fed to the computer of the medical instrument. Then the operator must enter his/her operator identification for starting the treatment.

### BRIEF DESCRIPTION OF THE DRAWINGS

Hereunder an embodiment of the invention is explained in detail with reference to the drawings, wherein these explanations may not be considered as limiting the scope of protection of the invention. The scope of protection is rather defined by the claims. In the drawings:

FIG. 1 shows a schematic representation of a network to which a plurality of medical instruments are connected, wherein each medical instrument comprises a read unit for a transponder, and

FIG. 2 shows a schematic representation of the data transmission between a transponder and the read unit.

### DETAILED DESCRIPTION OF THE PREFERRED EMBODIMENT OF THE INVENTION

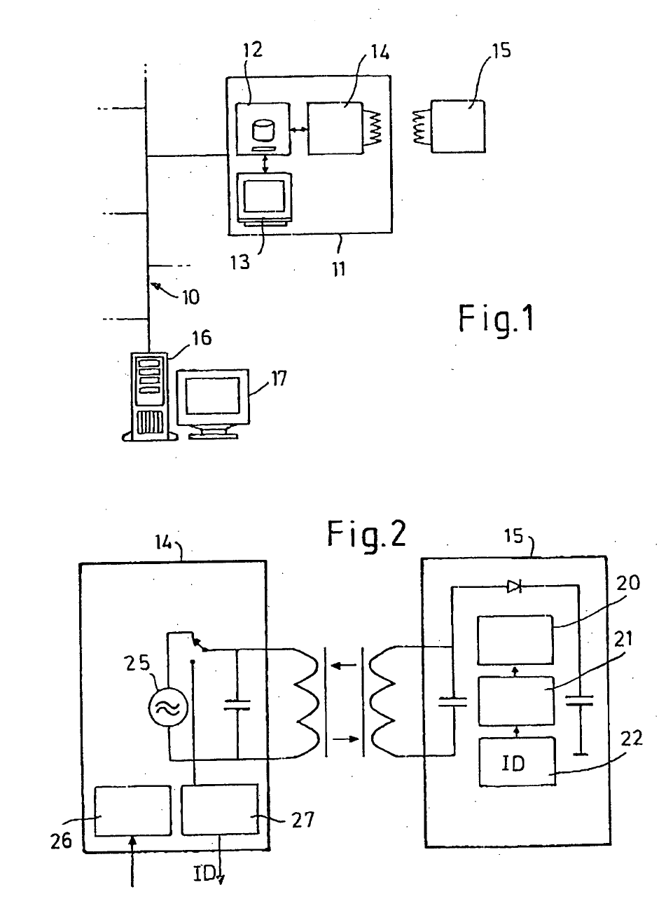

FIG. 1 shows a dialysis station comprising a data network 10 to which various medical instruments 11 are connected, which in this case are dialysis machines. Each medical instrument 11 comprises a computer 12 having a software and a data memory as well as an input/output device 13, the latter being a screen with touch screen function.

computer 12 has connected thereto a write/read unit 14 which is capable of contactlessly communicating with transponders 15. The transponders 15 comprise a radio tag, which communicates via radio waves with the write/read unit 14, and a data memory for storing received data. The communication between read unit and radio tag takes place bidirectionally.

The network 10 has further connected thereto a server 16 provided with a screen 17. The server 16 comprises a large data memory which can be accessed by the computers 12 of all instruments 11. The server 16, in turn, may be connected to a superordinate network, for example the internet or an intranet.

FIG. 2 shows the cooperation of the read unit 14 and the transponder 15. Both components communicate with each other via radio waves. The transponder 15 comprises a power source 20, a control unit 21 and a memory 22 which contains, inter alia, the operator identification ID of the owner of the transponder. The memory 22 can further be used for storing records.

The write/read unit 14 comprises an oscillator 25 for generating the carrier frequency for data transmission, and interfaces 26 und 27 for communication with the computer 12.

The write/read unit 14 recognizes a transponder 15 located in the vicinity of the read unit 14 and inquires about the identification ID stored in the transponder. In response to this inquiry, the transponder transmits the identification ID to the read unit. The identification ID includes information on the person concerned, namely whether the person belongs to the nursing personnel or to the technical service personnel. The two cases involve different access authorizations for modifying and/or setting programs and program parameters at the screen 13.

Further, there exist transponders 15 for patients. In this case, the identification ID includes information on the patient status of the owner. In the memory 22 further personal data of the owner can be stored, such as name, identification, weight and the like. Further, therapy values, such as treatment time, quantity of collected ultrafiltrate, conductivity of the haemodialysis solution etc. can be stored in the memory. At the end of the treatment, the treatment record produced by the computer 12 can be returned for storing in the transponder 15 of the operator and/or the transponder 15 of the patient. In this manner, it is possible to transmit the treatment record to a computer which is not connected to the data network 10.

In the computer 12 the access authorization for setting certain programs or program parameters is determined on the basis of the status information assigned to an identification ID. The status information states whether the person concerned belongs to the nursing personnel, the technical service personnel or any other group. The status information may be part of the identification ID. On the other hand, it is also possible that based on a list stored in the computer 12 or in the server 16 it is determined, on the basis of the identification ID, what status is assigned to the person concerned. Further, it is possible to check whether the person is allowed to be treated with the medical instrument involved. For this purpose, a personal authorization is included in the computer system.

The following table 1 shows an example of different access levels which are assigned to a respective status of a potential operator. The “setting-enabling data” show those settings the operator is authorized to carry out. When the instrument has determined the status of the operator, it automatically allows the corresponding settings to be carried out.

TABLE 1

| Access level | Setting enabling | Status |
|---|---|---|
| 0 | None, indication of act- | Patient |
| | ual values only | |
| 1 | Operator entries and | Nursing personnel |
| | alarm acknowledgement | |
| 2 | Treatment limit-values, | Physician |
| | operator entries and | |
| | alarm acknowledgement | |
| 3 | Machine configuration, | Technical service personnel |
| | service parameters, | |
| | treatment limit-values, | |
| | operator entries and | |
| | alarm acknowledgement | |

The term “machine configuration” includes information on accessory devices and additional capabilities of the machine, such as single-needle equipment. “Service parameters” are calibration values and machine-related limit values.

Although the invention has been described and illustrated with reference to specific illustrative embodiments thereof, it is not intended that the invention be limited to those illustrative embodiments. Those skilled in the art will recognize that variations and modifications can be made without departing from the true scope of the invention as defined by the claims that follow. It is therefore intended to include within the invention all such variations and modifications as fall within the scope of the appended claims and equivalents thereof.

1. Method for allowing operator entries at a medical instrument (11) which is suitable for carrying out a medical treatment of the body of a patient according to predetermined programs using selectable program parameters, wherein an operator identification (ID) is entered into a computer (12) controlling said medical instrument (11), and wherein, in dependence on the operator identification (ID), different access authorizations for selecting programs and/or modifying program parameters and/or entering alarm acknowledgements are granted.

2. Method according to claim 1, wherein patient data are stored in the computer (12), to which patient data the selected programs and program parameters are assigned and further the operator identification of the responsible operator is added, whereby a record on the treatment process is prepared and stored.

3. Method according to claim 1, wherein the operator identification (ID) is contained in a transponder (15) carried along by the operator, said transponder (15) contactlessly communicating with a read unit (14) of the medical instrument (11).

4. Method according to claim 2, wherein the record on the treatment process and information on the identity of the patient are transmitted to the transponder (15) and stored there.

5. Method according to claim 1, wherein a distinction is made between the medical nursing personnel and technical service personnel, and wherein only the operator identification (ID) of the service personnel allows for modification of the machine configuration and service parameters.

6. Method according to claim 2, wherein the medical instrument (11) comprises sensors whose measuring data are registered in the record on the treatment process.

7. Medical instrument, in particular blood treatment instrument, for extracorporeal blood treatment, comprising a computer (12) controlling the blood treatment, said computer (12) being capable of executing programs with selectable program parameters, wherein the access authorization with regard to selection of programs, program parameters and alarm acknowledgements is determined in dependence on an operator identification (ID) individually assigned to different operators.

8. Medical instrument according to claim 7, wherein the operator identification (ID) is contained in a transponder (15) which communicates with a read unit (14) of the medical instrument (11).

9. Medical instrument according to claim 8, wherein the transponder (15) comprises a memory (22) for storing data received from the read unit (14).
